(19) United States
(12) Patent Application Publication
(10) Pub. No.: US 2026/0159460 A1
(43) Pub. Date: Jun. 11, 2026

(54) INTEGRATED BIO-REFINERY FOR BIO-P-XYLENE WITH INTERNAL BIO-ETHYLENE PRODUCTION

(71) Applicant: UOP LLC, Des Plaines, IL (US)

(72) Inventors: Gregory B. Kuzmanich, Arlington Heights, IL (US); Dean E. Rende, Arlington Heights, IL (US); Steven L. Chmura, Des Plaines, IL (US)

(21) Appl. No.: 18/975,778

(22) Filed: Dec. 10, 2024

(51) Int. Cl. C07C 2/86; C12P 7/06

(52) U.S. Cl. CPC C07C 2/865; C12P 7/06; C07C 2527/182

(57) ABSTRACT

Integrated processes for producing p-xylene from biomass are described. The biomass is converted to sugars which are converted to 5-hydroxymethylfurfural (HMF) which is converted to dimethylfuran (DMF). The biomass derived DMF is reacted with internally generated de-watered, unpurified ethylene to form the p-xylene. The de-watered, unpurified ethylene is also derived from sugars made from the biomass.

### BACKGROUND

C₈ alkylaromatic hydrocarbons are generally considered to be valuable products, with a high demand for p-xylene. For example, p-xylene is used to commercially synthesize terephthalic acid, a raw material in the manufacture of polyester fabrics.

Major sources of p-xylene include mixed xylene streams that result from the refining of crude oil. Examples of such streams are those resulting from commercial xylene isomerization processes or from the separation of C₈ alkylaromatic hydrocarbon fractions derived from a catalytic reformate by liquid-liquid extraction and fractional distillation. The p-xylene may be separated from a p-xylene-containing feed stream, usually containing a mixture of all three xylene isomers, by crystallization and/or adsorptive separation.

Thus, most p-xylene is produced from petroleum-based feedstocks. However, producing p-xylene from petroleum-based feedstocks maintains reliance on refining petroleum and creates greenhouse gas emissions. A biobased p-xylene replacement from renewable feedstock would lower greenhouse gas (GHG) emissions and reduce reliance on petroleum resources. Not being bound by any theory, it is believed that bio- p-xylene may be carbon negative.

More recently, it has been suggested that p-xylene can be produced from a biomass derived component. For example, one process involves pyrolysis of biomass to pyrolysis oil which is a complex mixture of oxygenated hydrocarbons. The pyrolysis oil is hydrotreated to less than 10 ppm oxygen to form a bio-derived reformate, which can then be processed in a traditional aromatics complex to produce p-xylene. However, this process in not economical due to the low yields, issues with the pyrolysis oil fouling reactors, extremely difficult hydrodeoxygenation conditions needed to achieve the aromatics complex specification for oxygen content, and the general expense of producing p-xylene from a mixed reformate.

Producing p-xylene from sustainable sugar derived furans such as dimethylfuran (DMF) provides an alternative route to traditional petroleum-based production. However, currently, biochemicals derived from biomass are at a significant cost disadvantage compared to corresponding petroleum derived products due to the cost of the feedstocks (i.e., sugars).

Accordingly, there is a need for improved and cost effective processes to produce p-xylene from biomass.

### BRIEF DESCRIPTION OF THE DRAWINGS

FIG. 1 is an illustration of a proposed process for making p-xylene from biomass.

FIG. 2 is an illustration of one embodiment of an integrated process for the production of p-xylene from biomass according to the present invention.

### DESCRIPTION

The present invention meets this need by providing cost-effective, high yield processes for making p-xylene from biomass. The integrated processes produce p-xylene from biomass. The biomass is converted to sugars, which are converted to 5-hydroxymethylfurfural (HMF), which is converted to dimethylfuran (DMF). The processes use internally generated de-watered, unpurified ethylene which is also derived from sugars made from the biomass.

The processes have several benefits. They avoid feedstock costs for bio-ethylene, which is currently trading at almost the same price as p-xylene. They provide a high value end use for the biomass-derived C₅ sugar stream. In addition, the internally generated de-wateredethylene does not need to be purified before the DMF to p-xylene reaction, reducing the cost of production. Furthermore, the p-xylene produced by these processes is polymerization grade, thereby avoiding expensive purification processes, which further reduces the cost and GHG of the processes.

As used herein “biomass” includes, but is not limited to, lignin, plant parts, fruits, vegetables, plant processing waste, wood chips, chaff, grain, grasses, corn, corn husks, weeds, aquatic plants, hay, paper, recycled paper and paper products, agricultural and/or logging residues, forest timber, lumber, hemp, and/or construction debris (e.g., recycled wood) and any cellulose, lignin, or combinations thereof containing biological material or material of biological origin.

One aspect of the invention is an integrated process for the production of p-xylene. In one embodiment, the process comprises pretreating biomass in a pretreatment zone to produce pretreated biomass. The pretreated biomass is separated into at least a cellulosic pulp stream comprising cellulosic pulp, and a first soluble sugar stream comprising C₅₋₆ soluble sugars. At least a portion of the cellulosic pulp stream is contacted with a first acid catalyst to produce an HMF stream comprising 5-hydroxymethylfurfural (HMF). The HMF stream is hydrodeoxygenated with a hydrodeoxygenation catalyst to produce a DMF stream comprising dimethylfuran (DMF). The C₅₋₆ soluble sugars in at least a portion of the first soluble sugar stream are converted forming a de-watered, unpurified ethylene stream comprising ethylene. The DMF in the DMF stream is reacted with the ethylene in the de-watered, unpurified ethylene stream in the presence of a second acid catalyst to form p-xylene.

A variety of pretreatment steps could be performed on the biomass. For example, the pretreatment may comprise depolymerizing the lignin in the biomass to separate the lignin from the cellulosic pulp. Depolymerization can be performed using any suitable process. Suitable processes include, but are not limited to, solvolysis, steam explosion, sulfite pulping, hydrogenation, hydrogenolysis, alkaline pulping, or combinations thereof. Another pretreatment process is mechanically refining the biomass to reduce the particle size of the cellulosic pulp.

In some embodiments, the pretreated biomass is separated into at least a cellulosic pulp stream comprising cellulosic pulp, and a first soluble sugar stream comprising C₅₋₆ soluble sugars. In some embodiments, pretreated biomass is separated into at least a cellulosic pulp stream, a first soluble sugar stream, a lignin stream comprising lignin, and/or a waste stream comprising miscellaneous organic compounds.

In some embodiments, the first soluble sugar stream further comprises a lignin derived species, and the first soluble sugar stream is separated into a second soluble sugar stream comprising the C₅₋₆ soluble sugars and a lignin stream comprising the lignin derived species before converting the C₅₋₆ soluble sugars. In some embodiments, the first soluble sugar stream is separated into the second soluble sugar stream and the lignin stream using pH adjustment and precipitation, centrifugation, decanting, membrane filtration, liquid-liquid extractions, distillation, or combinations thereof. In some embodiments, the pretreated biomass is separated into at least the cellulosic pulp stream, the first soluble sugar stream, and a lignin stream comprising 50% or more of monomeric and oligomeric lignin derivatives with an average molecular weight of less than 2000 Da.

In some embodiments, the lignin stream comprises 50 wt % or more of oxygenated aromatic species with an average molecular weight of less than 2000 Da, and greater than 50 wt % by mass hydrogen and carbon atoms.

In some embodiments, the second soluble sugar stream is divided into a first portion and a second portion, and only a portion of the second soluble sugar stream is sent for conversion to ethylene. Alternatively, the whole second soluble sugar stream may be sent for conversion.

In some embodiments, the lignin stream is hydrodeoxygenated to form a hydrocarbon stream comprising C₁ to C₂₀ hydrocarbons, and the hydrocarbon stream is separated in a fractionation zone to form an aviation fuel stream comprising C₉ to C₁₆ hydrocarbons, or a naphtha stream comprising C₅ to C₈ hydrocarbons, or a diesel stream comprising C₉ to C₂₂ hydrocarbons, or combinations thereof.

In some embodiments, contacting the cellulosic pulp stream with a first acid catalyst comprises hydrolyzing the cellulosic pulp in the cellulosic pulp stream with a cellulase enzyme in a hydrolysis reaction zone comprising a hydrolysis reactor to produce a third soluble sugar stream comprising C₆ soluble sugars, and contacting the third soluble sugar stream with the first acid catalyst.

In some embodiments, the process further comprises contacting the cellulosic pulp with an aqueous liquid at temperatures above 50° C. with a residence time of 10 minutes or less before hydrolyzing the cellulosic pulp.

In some embodiments, the sugars from the hydrolysis reaction zone are contacted with the first acid catalyst to produce HMF. Any suitable acid catalyst for converting sugars to HMF can be used. In some embodiments, the first acid catalyst comprises a metal phosphate, wherein the first acid catalyst has a ratio of Bronsted acid sites to Lewis acid sites greater than or equal to 0.27 and a total acid density less than or equal to 0.4.

In some embodiments, the feed for the conversion of sugars to HMF comprises sugars produced in the hydrolysis reaction and/or sugars obtained from the separation of sugars and lignin. The sugars may comprise one or more sugar monomers and/or oligomers. In some embodiments, the feed comprises one or more sugar monomers and/or oligomers. The sugar oligomers may comprise oligosaccharides having between 3 and 10 sugar residues, or disaccharides, or combinations thereof. Sugar monomers are monosaccharides including, but not limited to, glucose, fructose, xylose, galactose, and the like, and their isomers.

In some embodiments, the metal phosphate comprises hafnium phosphate, or zirconium phosphate, or combinations thereof. In some embodiments, the metal phosphate has a mole ratio of phosphorus to metal in a range of 0.1:1 to 10:1.

The feed may be combined with water and optionally a solvent. When the solvent is present, the mole ratio of solvent to water may be in the range of 0.01:1 to 100:1. Suitable solvents include, but are not limited to, cyclic ethers, alcohols, sulfoxides, ketones, or combinations thereof. Suitable cyclic ethers include, but are not limited to, tetrahydrofuran, methyl tetrahydrofuran, dioxane, dimethylfuran, or combinations thereof. Suitable alcohols include, but are not limited to ethanol, butanol, and the like. Suitable sulfoxides include, but are not limited to, dimethyl sulfoxide. Suitable ketones include, but are not limited to, methyl isobutylketone, gamma valero lactone, or combinations thereof.

The water may include a salt. Typically, the mole ratio of the salt to the water is in the range of 0.001:1 to 0.5:1. Suitable solvents include, but are not limited to, sodium chloride, lithium chloride, potassium chloride, cesium chloride, magnesium chloride, calcium chloride, or combinations thereof.

The concentration of the feed in the water and solvent may be in the range of 0.01 wt % to 20 wt %.

Suitable operating conditions include, but are not limited to, temperatures in the range of 100° C. to 250° C., pressures in the range of 0 MPa to 6.9 MPa, or both. Suitable contact times include a range of 1 sec to 24 hr.

The process may comprise batch processes, continuous processes, or semi-continuous processes.

The process may take place in a single reactor or in multiple reactors (two or more). In some embodiments, the biomass-derived cellulose may be contacted with the metal phosphate catalyst in a first reactor, resulting in the biomass-derived cellulose being converted to 5-hydroxymethylfurfural and sugar monomers and/or oligomers. The sugar monomers and/or oligomers produced in the first reactor may be contacted with a second catalyst in a second reactor resulting in the sugar monomers and/or oligomers being converted to additional 5-hydroxymethylfurfural in the second reactor. In this arrangement, the first and second catalysts may be the same, or they may be different. Any suitable catalyst can be used for the second catalyst. Suitable catalysts include, but are not limited to, metal phosphates, aluminosilicate zeolites, silica aluminophosphate zeolites, zirconium sulfates, homogenous acids, metal oxides, or combinations thereof.

The catalyst comprising the metal phosphate and the second catalyst may optionally include a binder such as silica or alumina, as is well known in the art.

In some embodiments, the conversion of the biomass-derived cellulose and/or sugar monomers and/or oligomers is greater than or equal to 75%, or 80%, or 85%, or 90%. In some embodiments, the yield of 5-hydroxymethylfurfural is greater than or equal to 20%, or 25%, or 30%, or 35%, or 40%. In some embodiments, the conversion of the biomass-derived cellulose and/or sugar monomers and/or oligomers is greater than or equal to 75%, and the yield of 5-hydroxymethylfurfural is greater than or equal to 20%. In some embodiments, the conversion of the biomass-derived cellulose and/or sugar monomers and/or oligomers is greater than or equal to 90%, and the yield of 5-hydroxymethylfurfural is greater than or equal to 30%. In some embodiments, the conversion of the biomass-derived cellulose and/or sugar monomers and/oligomers is greater than or equal to 90%, and the yield of 5-hydroxymethylfurfural is greater than or equal to 35%. In some embodiments, the conversion of the biomass-derived cellulose and/or sugar monomers and/or oligomers is greater than or equal to 90%, and the yield of 5-hydroxymethylfurfural is greater than or equal to 40%.

Another aspect of the invention is a process for synthesizing 5-hydroxymethylfurfural. In one embodiment, the process comprises contacting a feed comprising biomass-derived cellulose, or a sugar monomer or oligomer, or a mixture thereof with a catalyst in the presence of water and a solvent, wherein the catalyst comprises a metal phosphate wherein the metal phosphate comprises hafnium phosphate, or zirconium phosphate, or combinations thereof, wherein the catalyst has a ratio of Bronsted acid sites to Lewis acid sites greater than or equal to 0.27 and a total acid density less than or equal to 0.4; and wherein the feed is contacted with the catalyst at a temperature in a range of 100° C. to 250° C., or at a pressure in a range of 0 MPa to 6.9 MPa, or for a time in a range of 1 sec to 24 hr, or combinations thereof.

In some embodiments, the process include one reactor, or two (or more) reactors. The biomass-derived cellulose feed can be contacting with the first catalyst in the first reactor where the biomass-derived cellulose is converted to 5-hydroxymethylfurfural and sugar monomers and/or oligomers. The sugar monomers and/or oligomers produced in the first reactor can be contacted with a second catalyst in the second reactor where the sugar monomers and/or oligomers are converted to additional 5-hydroxymethylfurfural. Sugar monomers contacted with the catalyst would be converted to 5-hydroxymethylfurfural, and any unconverted sugar monomers would be recycled.

This process is described in U.S. patent application Ser. No. 18/890,832, filed Sep. 20, 2024, entitled Process for Producing 5-Hydroxymethylfurfural Using Metal Phophates, which is incorporated herein by reference in its entirety.

The HMF is hydrodeoxygenated in the presence of a hydrodeoxygenation catalyst to produce a DMF stream comprising dimethylfuran (DMF). The hydrodeoxygenation reaction conditions include a temperature in the range of 20° C. to 300° C., or 100° C. to 200° C.; and pressures in the range of 70 psig to 1500 psig. Suitable hydrodeoxygenation catalysts include, but are not limited to, Co, Cu, Zn, Pd, Pt, Ni, Ru, Fe, W supported on alumina, silica, carbon, silica-alumina, titantia, or zirconia. The solvent desirably would be the one from the prior reaction without any intervening fractionation. Suitable solvents include, but are not limited to, cyclic ethers, alcohols, sulfoxides, ketones, or combinations thereof. Suitable cyclic ethers include, but are not limited to, tetrahydrofuran, methyl tetrahydrofuran, dioxane, dimethylfuran, or combinations thereof. Suitable alcohols include, but are not limited to ethanol, butanol, and the like. Suitable sulfoxides include, but are not limited to, dimethyl sulfoxide. Suitable ketones include, but are not limited to, methyl isobutylketone, gamma valero lactone, or combinations thereo

In some embodiments, the C₅₋₆ soluble sugars are converted to ethylene by fermenting the C₅₋₆ soluble sugars to ethanol, which is dehydrated to form ethylene. The ethylene is subsequently dewatered to produce a dewatered, unpurified ethylene stream. Alternatively, a metal/acid catalyst could be used to convert the sugars to low molecular weight alcohols, but the selectivity to ethanol is not high.

In some embodiments, the catalyst for dehydrating the ethanol comprises activated alumina, aluminosilicate zeolite, silicoaluminophosphate or combinations thereof. In some embodiments, the reaction conditions for dehydrating the ethanol include a temperature in a range of 125-400° C. and a residence time of less than 10 minutes.

In some embodiments, the de-watered, unpurified ethylene stream comprises less than 1 wt % CO and CO₂, or less than 5 wt % butenes, or less than 1000 ppm ethers, or less than 1000 ppm carbonyls, or combinations thereof.

In some embodiments, the DMF and the de-watered, unpurified ethylene are reacted in the presence of a second acid catalyst to form p-xylene. The second acid catalyst has a relatively low silica to alumina ratio of around 25 compared with conventional catalysts which have a ratio greater than 1000. The catalyst has phosphorus and may be 12 membered ring zeolite such as a beta zeolite. The catalyst can be used to generate a high yield of bio-based p-xylene.

The reaction of biomass derived DMF and the de-watered, unpurified ethylene proceeds in the presence of the second acid catalyst as discussed above under suitable cycloaddition reaction conditions. A molar ratio of ethylene to DMF may be in the range of 1:100 to 100:1, or 1:50 to 50:1, or 1:10 to 10:1, or 1:1 to 2:1. A weight ratio of catalyst to the DMF may be in the range of 0.001:1 to 10:1, or 0.01:1 to 10:1.

One or more of the reactants may be in another liquid, such as a hydrocarbon oil.

Suitable temperatures in the reactor or reaction zone in which the catalyst is disposed (e.g., in a batch reactor or as a fixed or moving bed in a continuous reaction system) are in the range of 100 to 500° C., or 200° C. to 300° C., or 150° C. to 225° C. Favorable cycloalkylation reaction conditions also include a reaction pressure in the range of 689 kPa to 17,237 kPa (100 to 2,500 psig), or 1,379 kPa to 13,790 kPa (200 to 2,000 psig).

Any suitable acid catalyst for the reaction of DMF and ethylene can be used. In some embodiments, the second acid catalyst comprises phosphorus and a silicon and aluminum oxide zeolite having a silicon to aluminum ratio of less than 1:1000. In some embodiments, the second acid catalyst comprises a beta zeolite with a silicon oxide to aluminum oxide molar ratio in a range of 1:1 and 1:25 and a phosphorus content of 0.001 to 10 wt %.

Whether the reaction is carried out batchwise or continuously, the cycloaddition reaction conditions also generally include a reactor residence time in the range from about 0.1 second to about 48 hours, or from about 0.05 hours to about 30 hours, or from 0.1 to 10 hours. The reactor residence time may be optimized for multiple factors, including for example, maximizing p-xylene selectivity the expense of conversion which can be done by recycling the unreacted feed after removing the product. The biomass derived compound may be continuously fed to a cycloaddition reaction zone, for example, at a liquid hourly space velocity (LHSV) in the range of 0.05 hr⁻¹ to 5 hr⁻¹. As is understood in the art, the Liquid Hourly Space Velocity (LHSV, expressed in units of hr⁻¹) is the volumetric liquid flow rate over the catalyst bed divided by the bed volume and represents the equivalent number of catalyst bed volumes of liquid processed per hour. The LHSV is therefore closely related to the inverse of the reactor residence time.

In an exemplary continuous process, the reactants are continuously fed to one or more reactors containing a catalyst which may be a CSTR type reactor (stirred tank) or which includes a single or multiple fixed beds of the catalyst. The multiple fixed beds may be in a parallel swing-bed reactor configuration or in series in a lead-lag configuration. The fixed beds may occasionally be taken offline for catalyst regeneration. A product comprising the converted p-xylene is continuously withdrawn together with unconverted reactants and reaction byproducts such as 2,5-hexanedione. The unconverted materials are preferably separated, for example, based on differences in their relative volatility using one or more separation operations (e.g., flash separation or distillation) employing a single stage or multiple stages of vapor-liquid equilibrium contacting. In some cases, it may be desirable to convert 2,5-hexandedione, which is a hydration byproduct of DMF, back to DMF to improve product yields.

Additionally, unreacted components may be recycled back to the feed, without or without additional drying to remove water. The drying or reduction of water content, for at least the recycle, will reduce the formation of hexanedione and reduce side reactions cause by the presence of water during the dehydration reaction. Drying may be performed via distillation or a molecular sieve.

This process is described in U.S. patent application Ser. No. 18/459,612, filed Sep. 1, 2023, entitled Processes for Synthesizing Paraxylene and Catalyst for Same, which is incorporated herein by reference in its entirety.

Another aspect of the invention is an integrated process for the production of p-xylene. In one embodiment, the process comprises: pretreating biomass in a pretreatment zone to produce pretreated biomass; separating the pretreated biomass into at least a cellulosic pulp stream comprising cellulosic pulp, a first soluble sugar stream comprising C₅₋₆ soluble sugars, and a lignin stream comprising lignin-derived species; hydrolyzing the cellulosic pulp with a cellulase enzyme in a hydrolysis reaction zone comprising a hydrolysis reactor to produce a second soluble sugar stream comprising C₆ soluble sugars; contacting the second soluble sugar stream and optionally a portion of the first soluble sugar stream with a first acid catalyst to produce an HMF stream comprising 5-hydroxymethylfurfural (HMF); hydrodeoxygenating the HMF stream with a hydrodeoxygenation catalyst to produce a DMF stream comprising dimethylfuran (DMF); converting the C₅₋₆ soluble sugars in at least a portion of the first soluble sugar stream forming a de-watered, unpurified ethylene stream comprising ethylene; and reacting the DMF in the DMF stream with the ethylene in the de-watered, unpurified ethylene stream in the presence of a second acid catalyst to form p-xylene. The p-xylene is of sufficient purity to be used in downstream applications without further purification from the other xylene isomers and ethylbenzene.

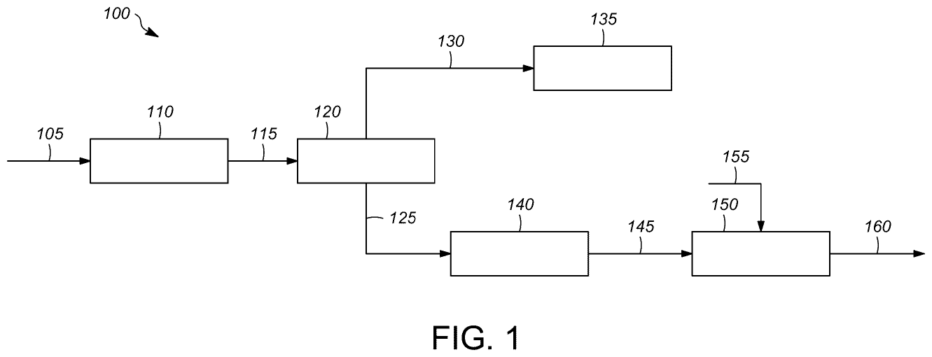

FIG. 1 is an illustration of an existing process 100 for making p-xylene from biomass. The biomass feedstream 105 is sent to a pretreatment zone 110.

The pretreated biomass stream 115 is separated in a first separation zone 120 into a cellulosic pulp stream 125 comprising cellulosic pulp and waste stream 130 comprising lignin derived species, soluble sugars, and miscellaneous soluble organic compounds. Most refineries do not have an economical route to upgrading the soluble sugar mixture containing C₅₋₆ sugars and lignin, and the waste stream 130 is either burned in biomass burner 135 or used as animal feed.

The cellulosic pulp stream 125 is enzymatically hydrolyzed to sugar in a hydrolysis reaction zone 140 which may include an isomerization and purification units to convert the hydrolyzed sugar to specific isomers such as fructose. The sugar stream 145 is sent to a multistage reaction zone 150 where the sugars are converted to HMF, the HMF is converted to DMF, and the DMF is reacted with purified ethylene from a purified ethylene stream 155 to produce p-xylene stream 160.

This process has several disadvantages. Purified bio-ethylene is expensive, and it is not commercially available at the present time. In addition, components in the waste stream that could be converted to other materials are burned.

It has been surprisingly found that purified ethylene is not required to produce p-xylene. De-watered, unpurified ethylene produced from sugars derived from the biomass can be used without purification, reducing the cost of the ethylene component and reducing waste from the process. In addition, lignin can be produced and converted to useful hydrocarbons.

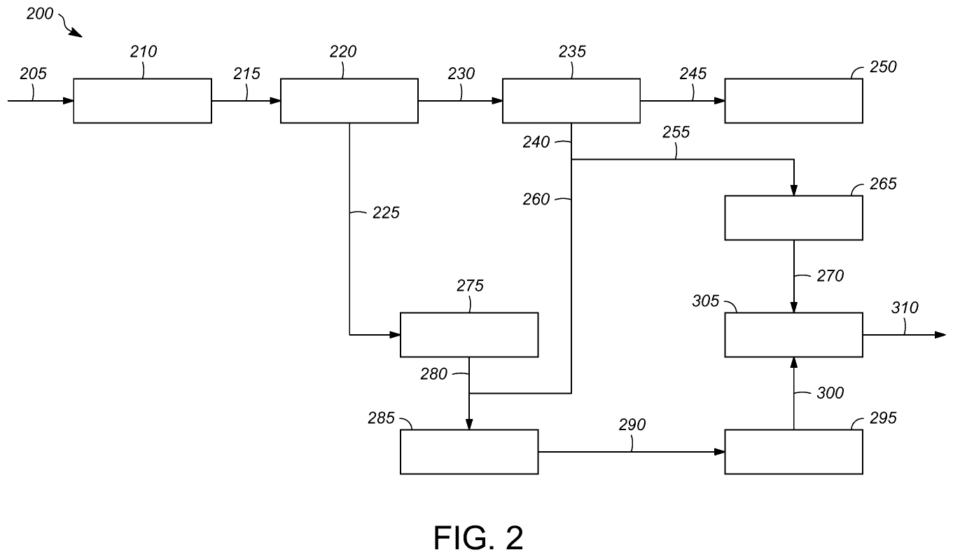

FIG. 2 is an illustration of one embodiment of a process 200 for the production of p-xylene from biomass according the present invention. The biomass feed stream 205 is sent to a pretreatment zone 210.

The pretreated biomass stream 215 is separated in a first separation zone 220 into a cellulosic pulp stream 225 comprising cellulosic pulp and a first soluble sugar stream 230 comprising C₅₋₆ soluble sugars. The first soluble sugar stream 230 may also include lignin. The separation may include an organic stream comprising organics (not shown). Any suitable separation process can be used for the separation, including, but not limited to, filter press, centrifuge, dewatering, screw press, decanter, membrane filtration, and the like, and combinations thereof.

The first soluble sugar stream 230 is sent to a second separation zone 235 where it is separated into a second soluble sugar stream 240 and a lignin stream 245 comprising lignin derived species. Lignin derived species are primarily aromatic compounds that are derived from the three monolignols: p-coumaryl, coniferyl, and sinapyl. These lignin derived species contain at least one aromatic functional group and may be fragments of, or oligomers of, the three primary monolignols. The lignin stream may comprise 50% or more of monomeric and oligomeric lignin derivatives with an average molecular weight of less than 2000 Da. The separation may be performed using any suitable separation process including, but not limited to, pH adjustment and precipitation, centrifugation, decanting, membrane filtration, distillation, or combinations thereof.

The lignin stream may comprise 50% or more of monomeric and oligomeric lignin derivatives with an average molecular weight of less than 2000 Da. The lignin stream 245 may be sent to a hydrodeoxygenation reaction zone 250 comprising a hydrodeoxygenation reactor and fractionation section where it is hydrodeoxygenated to form a hydrocarbon stream comprising C₁ to C₂₀ hydrocarbons. The heteroatom containing lignin derivatives are hydrodeoxygenated to hydrocarbons with less than 0.3 wt % oxygen using a heterogeneous bifunctional catalyst comprising an acid and metal function or steam. The fractionation section may comprise one or more fractionation columns to separate off any light fractions that form (e.g., water and C₄ hydrocarbons) and to separate the hydrocarbon products by boiling point. The feeds and any co-solvents may be recycled to the hydrodeoxygenation reactor. The hydrocarbon stream is separated into one or more of an aviation fuel stream comprising C₈ to C₁₄ hydrocarbons, or a gas stream comprising C₆ to C₈ hydrocarbons, or a diesel stream comprising C₁₄ to C₂₂ hydrocarbons, or combinations thereof, for example. Other separations are known to those of skill in the art.

The second soluble sugar stream 240 may be divided into a first portion 255 and a second portion 260. The first portion 255 is sent to a sugar conversion zone 265 where the C₅₋₆ soluble sugars are converted to ethanol. Alternatively, all of the second soluble sugar stream 240 may be sent to the sugar conversion zone 265 or the second sugar conversion reaction zone 285. One method of converting the C₅₋₆ soluble sugars to ethanol is by fermentation.

The ethanol is dehydrated to form ethylene. Catalysts for the dehydration of the ethanol include, but are not limited to activated alumina, aluminosilicate zeolite, silicoaluminophosphate or combinations thereof. The silicoaluminophosphate or aluminosilicate zeolite may comprise an 8 or 10 membered ring pore structure. Reaction conditions for the dehydration include a temperature in a range of 125-400° C. and a residence time of less than 10 minutes.

The de-watered, unpurified ethylene stream 270 comprising ethylene, and less than 1 wt % CO and CO₂, or less than 5 wt % butenes, or less than 1000 ppm ethers, or less than 1000 ppm carbonyls, or combinations thereof.

The cellulosic pulp stream 225 is hydrolyzed with a cellulase enzyme in a hydrolysis reaction zone 275 comprising a hydrolysis reactor to produce a third soluble sugar stream 280 comprising C₆ soluble sugars.

The cellulosic pulp stream 225 may be contacted with an aqueous liquid at temperatures above 50° C. with a residence time of 10 minutes or less before hydrolyzing the cellulosic pulp, if desired.

The third soluble sugar stream 280 and the second portion 260 of the second soluble sugar stream 240 (if present) are sent to a second sugar conversion reaction zone 285 where the C₆ soluble sugars are contacted with a first acid catalyst and converted to HMF. The first acid catalyst may comprise a metal phosphate having a ratio of Bronsted acid sites to Lewis acid sites greater than or equal to 0.27 and a total acid density less than or equal to 0.4.

The HMF stream 290 from the second sugar conversion reaction zone 285 is sent to the hydrodeoxygenation reaction zone 295 and hydrodeoxygenated to form dimethylfuran (DMF).

The DMF stream 300 and the de-watered, unpurified ethylene stream 270 are sent to the p-xylene reaction zone 305 where they react in the presence of a second acid catalyst to form p-xylene. Any suitable acid catalyst may be used. The second acid catalyst may comprise phosphorus and a silicon and aluminum oxide zeolite having a silicon to aluminum ratio of less than 1:1000.

The p-xylene product stream 310 can be recovered.

### EXAMPLES

### Comparative Example

In a typical experiment, 12 wt % DMF in octane with ethylene (1.62 mole ratio ethylene:DMF) were loaded into an autoclave with a phosphorus containing beta zeolite (0.05 weight ratio catalyst:DMF). The reaction mixture was heated to 285° C. for 6 hours. The product was filtered to separate the catalyst from the liquid product.

The same phosphorus containing beta zeolite catalyst was used for all of the examples. Further details concerning the catalyst may be found in US Application Serial No. 2024/0208883, which is incorporated herein in its entirety.

### Example 1

12 wt % DMF in octane, 0.1 wt % (1000 ppm) ethyl acetate, and ethylene (1.62 mole ratio ethylene:DMF) were loaded into an autoclave with a phosphorus containing beta zeolite (0.05 weight ratio catalyst:DMF). The reaction mixture was heated to 285° C. for 6 hours. The product was filtered to separate the catalyst from the liquid product.

### Example 2

12 wt % DMF in octane, 0.1 wt % (1000 ppm) diethyl ether, and ethylene (1.62 mole ratio ethylene:DMF) were loaded into the autoclave with a phosphorus containing beta zeolite (0.05 weight ratio catalyst:DMF). The reaction mixture was heated to 285° C. for 6 hours. The product was filtered to separate the catalyst from the liquid product.

### Example 3

12 wt % DMF in octane, 0.005 wt % (50 ppm) acetaldehyde, and ethylene (1.62 mole ratio ethylene:DMF) were loaded into the autoclave with a phosphorus containing beta zeolite (0.05 weight ratio catalyst:DMF). The reaction mixture was heated to 285° C. for 6 hours. The product was filtered to separate the catalyst from the liquid product.

### Example 4

12 wt % DMF in octane with a gas blend containing ethylene (1.62 mole ratio ethylene:DMF) and carbon dioxide (2000 ppm) were loaded into the autoclave with a phosphorus containing beta zeolite (0.05 weight ratio catalyst:DMF). The reaction mixture was heated to 285° C. for 6 hours. The product was filtered to separate the catalyst from the liquid product.

### Example 5

12 wt % DMF in octane with a gas blend containing ethylene (1.62 mole ratio ethylene:DMF) and butenes (1000 ppm) were loaded into the autoclave with a phosphorus containing beta zeolite (0.05 weight ratio catalyst:DMF). The reaction mixture was heated to 285° C. for 6 hours. The product was filtered to separate the catalyst from the liquid product.

To calculate conversion and selectivity, hexanedione (HDO) was considered as unreacted DMF in the product due to the equilibrium hydration/dehydration reaction of DMF and HDO. The following equations were used:

$$Conversion ( \% ) = 100 - \frac{( C unconverted DMF ( wt \% ) + C HDO ( wt \% ) )}{C Reactant DMF ( wt \% )} \times 100 \qquad EQ . 1$$

$$pX \frac{selectivity}{total} products ( \% ) = \frac{C Product species ( wt \% )}{\sum unconverted reactants in product + C HDO} \times 100 \qquad EQ . 2$$

$$pX yield = \frac{( DMF conversion ( \% ) x pX selectivity / total products ( \% ) )}{100} \qquad EQ . 3$$

| | Comparative | | | | | |
|---|---|---|---|---|---|---|
| Example | Example | 1 | 2 | 3 | 4 | 5 |
| Contaminant | | 0.1 | 0.1 | 0.2 | 0.1 | 0.005 |
| wt % | | | | | | |
| DMF | 61 | 57 | 58 | 61 | 59 | 61 |
| Conversion, % | | | | | | |
| pX | 85 | 85 | 85 | 85 | 84 | 85 |
| Selectivity, % | | | | | | |

As the examples show, the DMF conversion and p-xylene selectivity were not impacted at the contaminant levels tested.

### SPECIFIC EMBODIMENTS

While the following is described in conjunction with specific embodiments, it will be understood that this description is intended to illustrate and not limit the scope of the preceding description and the appended claims.

A first embodiment of the invention is a process for the production of p-xylene comprising pretreating biomass in a pretreatment zone to produce pretreated biomass; separating the pretreated biomass into at least a cellulosic pulp stream comprising cellulosic pulp, and a first soluble sugar stream comprising C₅₋₆ soluble sugars; contacting at least a portion of the cellulosic pulp stream with a first acid catalyst to produce an HMF stream comprising 5-hydroxymethylfurfural (HMF); hydrodeoxygenating the HMF stream with a hydrodeoxygenation catalyst to produce a DMF stream comprising dimethylfuran (DMF); converting the C₅₋₆ soluble sugars in at least a portion of the first soluble sugar stream forming a de-watered, unpurified ethylene stream comprising ethylene; reacting the DMF in the DMF stream with the ethylene in the de-watered, unpurified ethylene stream in the presence of a second acid catalyst to form p-xylene. An embodiment of the invention is one, any or all of prior embodiments in this paragraph up through the first embodiment in this paragraph wherein pretreating the biomass comprises depolymerizing lignin in the biomass to separate the lignin from the cellulosic pulp. An embodiment of the invention is one, any or all of prior embodiments in this paragraph up through the first embodiment in this paragraph wherein depolymerizing the lignin comprises depolymerizing the lignin by solvolysis, sulfite pulping, hydrogenation, hydrogenolysis, alkaline pulping, or combinations thereof. An embodiment of the invention is one, any or all of prior embodiments in this paragraph up through the first embodiment in this paragraph pretreating the biomass by mechanically refining the cellulosic pulp to reduce the particle size of the cellulosic pulp before contacting the cellulose pulp stream with the first acid catalyst. An embodiment of the invention is one, any or all of prior embodiments in this paragraph up through the first embodiment in this paragraph wherein the first soluble sugar stream further comprises a lignin derived species, further comprising separating the first soluble sugar stream into a second soluble sugar stream comprising the C₅₋₆ soluble sugars and a lignin stream comprising the lignin derived species before converting the C₅₋₆ soluble sugars. An embodiment of the invention is one, any or all of prior embodiments in this paragraph up through the first embodiment in this paragraph wherein the lignin stream comprises 50 wt % or more of oxygenated aromatic species with an average molecular weight of less than 2000 Da, and greater than 50 wt % by mass hydrogen and carbon atoms. An embodiment of the invention is one, any or all of prior embodiments in this paragraph up through the first embodiment in this paragraph wherein separating the first soluble sugar stream into the second soluble sugar stream and the lignin stream comprises separating the first soluble sugar stream into the second soluble sugar stream and the lignin stream using pH adjustment and precipitation, centrifugation, decanting, membrane filtration, distillation, or combinations thereof. An embodiment of the invention is one, any or all of prior embodiments in this paragraph up through the first embodiment in this paragraph further comprising dividing the second soluble sugar stream into a first portion and a second portion; wherein converting the C₅₋₆ soluble sugars in at least a portion of the first soluble sugar stream comprises converting the C₅₋₆ soluble sugars in the first portion of the second soluble sugar stream. An embodiment of the invention is one, any or all of prior embodiments in this paragraph up through the first embodiment in this paragraph further comprising hydrodeoxygenating the lignin derived species in the lignin stream to form a hydrocarbon stream comprising C₁ to C₂₀ hydrocarbons; and separating the hydrocarbon stream in a fractionation zone comprising a fractionation column to form an aviation fuel stream comprising C₉ to C₁₆ hydrocarbons, or a naphtha stream comprising C₅ to C₈ hydrocarbons, or a diesel stream comprising C₉ to C₂₂ hydrocarbons, or combinations thereof. An embodiment of the invention is one, any or all of prior embodiments in this paragraph up through the first embodiment in this paragraph wherein the lignin derived species contain a heteroatom, and wherein hydrodeoxygenating the lignin derived species comprises hydrodeoxygenating the lignin derived species to C₁ to C₂₀ hydrocarbons having less than 0.3 wt % oxygen using a heterogeneous bifunctional catalyst comprising an acid and metal function or steam. An embodiment of the invention is one, any or all of prior embodiments in this paragraph up through the first embodiment in this paragraph wherein separating the pretreated biomass comprises separating the pretreated biomass into at least the cellulosic pulp stream comprising cellulosic pulp, the first soluble sugar stream comprising C₅₋₆ soluble sugars, and a lignin stream comprising 50% or more of monomeric and oligomeric lignin derivatives with an average molecular weight of less than 2000 Da. An embodiment of the invention is one, any or all of prior embodiments in this paragraph up through the first embodiment in this paragraph wherein contacting at least the portion of the cellulosic pulp stream with a first acid catalyst comprises hydrolyzing the cellulosic pulp in the cellulosic pulp stream with a cellulase enzyme in a hydrolysis reaction zone comprising a hydrolysis reactor to produce a third soluble sugar stream comprising C₆ soluble sugars; and contacting the third soluble sugar stream with the first acid catalyst. An embodiment of the invention is one, any or all of prior embodiments in this paragraph up through the first embodiment in this paragraph where the de-watered, unpurified ethylene stream comprises less than 1 wt % CO and CO₂, or less than 5 wt % butenes, or less than 1000 ppm ethers, or less than 1000 ppm carbonyls, or combinations thereof. An embodiment of the invention is one, any or all of prior embodiments in this paragraph up through the first embodiment in this paragraph wherein converting the C₅₋₆ soluble sugars comprises fermenting the C₅₋₆ soluble sugars to ethanol; and dehydrating the ethanol to form the de-watered, unpurified ethylene stream. An embodiment of the invention is one, any or all of prior embodiments in this paragraph up through the first embodiment in this paragraph wherein a catalyst for dehydrating the ethanol comprises activated alumina, aluminosilicate zeolite, silicoaluminophosphate or combinations thereof, and wherein dehydrating the ethanol takes place at a temperature in a range of 125-400° C. and a residence time of less than 10 minutes. An embodiment of the invention is one, any or all of prior embodiments in this paragraph up through the first embodiment in this paragraph where the silicoaluminophosphate or aluminosilicate zeolite comprises an 8 or 10 membered ring pore structure. An embodiment of the invention is one, any or all of prior embodiments in this paragraph up through the first embodiment in this paragraph wherein the first acid catalyst comprises a metal phosphate, wherein the first acid catalyst has a ratio of Bronsted acid sites to Lewis acid sites greater than or equal to 0.27 and a total acid density less than or equal to 0.4. An embodiment of the invention is one, any or all of prior embodiments in this paragraph up through the first embodiment in this paragraph wherein the second acid catalyst comprises phosphorus and a silicon and aluminum oxide zeolite having a silicon to aluminum ratio of less than 1:1000. An embodiment of the invention is one, any or all of prior embodiments in this paragraph up through the first embodiment in this paragraph wherein the second acid catalyst comprises a beta zeolite with a silicon oxide to aluminum oxide molar ratio in a range of 11 and 125 and a phosphorus content of 0.001 to 10 wt %.

A second embodiment of the invention is a process for the production of p-xylene comprising pretreating biomass in a pretreatment zone to produce pretreated biomass; separating the pretreated biomass into at least a cellulosic pulp stream comprising cellulosic pulp, a first soluble sugar stream comprising C₅₋₆ soluble sugars, and a lignin stream comprising lignin-derived species; hydrolyzing the cellulosic pulp with a cellulase enzyme in a hydrolysis reaction zone comprising a hydrolysis reactor to produce a second soluble sugar stream comprising C₆ soluble sugars; contacting the second soluble sugar stream and optionally a portion of the first soluble sugar stream with a first acid catalyst to produce an HMF stream comprising 5-hydroxymethylfurfural (HMF); hydrodeoxygenating the HMF stream with a hydrodeoxygenation catalyst to produce a DMF stream comprising dimethylfuran (DMF); converting the C₅₋₆ soluble sugars in at least a portion of the first soluble sugar stream forming a de-watered, unpurified ethylene stream comprising ethylene; reacting the DMF in the DMF stream with the ethylene in the de-watered, unpurified ethylene stream in the presence of a second acid catalyst to form p-xylene.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. An integrated process for the production of p-xylene comprising:
pretreating biomass in a pretreatment zone to produce pretreated biomass;
separating the pretreated biomass into at least a cellulosic pulp stream comprising cellulosic pulp, and a first soluble sugar stream comprising C₅₋₆ soluble sugars;
contacting at least a portion of the cellulosic pulp stream with a first acid catalyst to produce an HMF stream comprising 5-hydroxymethylfurfural (HMF);
hydrodeoxygenating the HMF stream with a hydrodeoxygenation catalyst to produce a DMF stream comprising dimethylfuran (DMF);
converting the C₅₋₆ soluble sugars in at least a portion of the first soluble sugar stream forming a de-watered, unpurified ethylene stream comprising ethylene; and
reacting the DMF in the DMF stream with the ethylene in the de-watered, unpurified ethylene stream in the presence of a second acid catalyst to form p-xylene.

2. The process of claim 1 wherein pretreating the biomass comprises depolymerizing lignin in the biomass to separate the lignin from the cellulosic pulp.

3. The process of claim 2 wherein depolymerizing the lignin comprises depolymerizing the lignin by solvolysis, sulfite pulping, hydrogenation, hydrogenolysis, alkaline pulping, or combinations thereof.

4. The process of claim 1 pretreating the biomass by mechanically refining the cellulosic pulp to reduce the particle size of the cellulosic pulp before contacting the cellulose pulp stream with the first acid catalyst.

5. The process of claim 1 wherein the first soluble sugar stream further comprises a lignin derived species, further comprising:
separating the first soluble sugar stream into a second soluble sugar stream comprising the C₅₋₆ soluble sugars and a lignin stream comprising the lignin derived species before converting the C₅₋₆ soluble sugars.

6. The process of claim 5 wherein the lignin stream comprises 50 wt % or more of oxygenated aromatic species with an average molecular weight of less than 2000 Da, and greater than 50 wt % by mass hydrogen and carbon atoms.

7. The process of claim 5 wherein separating the first soluble sugar stream into the second soluble sugar stream and the lignin stream comprises separating the first soluble sugar stream into the second soluble sugar stream and the lignin stream using pH adjustment and precipitation, centrifugation, decanting, membrane filtration, distillation, or combinations thereof.

8. The process of claim 5 further comprising:
dividing the second soluble sugar stream into a first portion and a second portion;
wherein converting the C₅₋₆ soluble sugars in at least a portion of the first soluble sugar stream comprises converting the C₅₋₆ soluble sugars in the first portion of the second soluble sugar stream.

9. The process of claim 5 further comprising:
hydrodeoxygenating the lignin derived species in the lignin stream to form a hydrocarbon stream comprising C₁ to C₂₀ hydrocarbons; and
separating the hydrocarbon stream in a fractionation zone comprising a fractionation column to form an aviation fuel stream comprising C₉ to C₁₆ hydrocarbons, or a naphtha stream comprising C₅ to C₈ hydrocarbons, or a diesel stream comprising C₉ to C₂₂ hydrocarbons, or combinations thereof.

10. The process of claim 9 wherein the lignin derived species contain a heteroatom, and wherein hydrodeoxygenating the lignin derived species comprises:
hydrodeoxygenating the lignin derived species to C₁ to C₂₀ hydrocarbons having less than 0.3 wt % oxygen using a heterogeneous bifunctional catalyst comprising an acid and metal function or steam.

11. The process of claim 1 wherein separating the pretreated biomass comprises:
separating the pretreated biomass into at least the cellulosic pulp stream comprising cellulosic pulp, the first soluble sugar stream comprising C₅₋₆ soluble sugars, and a lignin stream comprising 50% or more of monomeric and oligomeric lignin derivatives with an average molecular weight of less than 2000 Da.

12. The process of claim 1 wherein contacting at least the portion of the cellulosic pulp stream with a first acid catalyst comprises:
hydrolyzing the cellulosic pulp in the cellulosic pulp stream with a cellulase enzyme in a hydrolysis reaction zone comprising a hydrolysis reactor to produce a third soluble sugar stream comprising C₆ soluble sugars; and
contacting the third soluble sugar stream with the first acid catalyst.

13. The process of claim 1 where the de-watered, unpurified ethylene stream comprises less than 1 wt % CO and CO₂, or less than 5 wt % butenes, or less than 1000 ppm ethers, or less than 1000 ppm carbonyls, or combinations thereof.

14. The process of claim 1 wherein converting the C₅₋₆ soluble sugars comprises:
fermenting the C₅₋₆ soluble sugars to ethanol; and
dehydrating the ethanol to form the de-watered, unpurified ethylene stream.

15. The process of claim 14 wherein a catalyst for dehydrating the ethanol comprises activated alumina, aluminosilicate zeolite, silicoaluminophosphate or combinations thereof, and wherein dehydrating the ethanol takes place at a temperature in a range of 125-400° C. and a residence time of less than 10 minutes.

16. The process of claim 15 where the silicoaluminophosphate or aluminosilicate zeolite comprises an 8 or 10 membered ring pore structure.

17. The process of claim 1 wherein the first acid catalyst comprises a metal phosphate, wherein the first acid catalyst has a ratio of Bronsted acid sites to Lewis acid sites greater than or equal to 0.27 and a total acid density less than or equal to 0.4.

18. The process of claim 1 wherein the second acid catalyst comprises phosphorus and a silicon and aluminum oxide zeolite having a silicon to aluminum ratio of less than 1:1000.

19. The process of claim 18 wherein the second acid catalyst comprises a beta zeolite with a silicon oxide to aluminum oxide molar ratio in a range of 1:1 and 1:25 and a phosphorus content of 0.001 to 10 wt %.

20. An integrated process for the production of p-xylene comprising:
pretreating biomass in a pretreatment zone to produce pretreated biomass;
separating the pretreated biomass into at least a cellulosic pulp stream comprising cellulosic pulp, a first soluble sugar stream comprising C₅₋₆ soluble sugars, and a lignin stream comprising lignin-derived species;
hydrolyzing the cellulosic pulp with a cellulase enzyme in a hydrolysis reaction zone comprising a hydrolysis reactor to produce a second soluble sugar stream comprising C₆ soluble sugars;
contacting the second soluble sugar stream and optionally a portion of the first soluble sugar stream with a first acid catalyst to produce an HMF stream comprising 5-hydroxymethylfurfural (HMF);
hydrodeoxygenating the HMF stream with a hydrodeoxygenation catalyst to produce a DMF stream comprising dimethylfuran (DMF);
converting the C₅₋₆ soluble sugars in at least a portion of the first soluble sugar stream forming a de-watered, unpurified ethylene stream comprising ethylene; and
reacting the DMF in the DMF stream with the ethylene in the de-watered, unpurified ethylene stream in the presence of a second acid catalyst to form p-xylene.
